(19) United States
(12) Patent Application Publication
(10) Pub. No.: US 2022/0226207 A1
(43) Pub. Date: Jul. 21, 2022

(54) TEMPORARY TATTOO METHOD

(71) Applicants: Selim Cengiç, Sariyer/Istanbul (TR); Mustafa Kemal YALÇINKAYA, Sisli/Istanbul (TR)

(72) Inventor: Mustafa Kemal Yalçinkaya, Istanbul (TR)

(73) Assignee: Selim Cengiç, Sariyer/Istanbul (TR)

(21) Appl. No.: 17/615,576

(22) PCT Filed: Jan. 20, 2021
(86) PCT No.: PCT/TR2021/050040

§ 371 Date: Nov. 30, 2021

(30) Foreign Application Priority Data
Nov. 10, 2020 (TR) 2020/17952

(51) Int. Cl. A61K 8/04; A61Q 1/02

(52) U.S. Cl. CPC A61K 8/046; A61K 2800/434; A61Q 1/025

(57) ABSTRACT

The present invention relates to the temporary tattoo method applied on human body, furniture, and vehicles by use of neon paint that is visible at night and is not harmful for human health by way of spraying.

### TECHNICAL FIELD

The present invention relates to the temporary tattoo method applied on human body, furniture, and vehicles by use of neon paint that is visible at night and is not harmful for human health by way of spraying.

### BACKGROUND ART

Tattoo is the general word for signs and designs made on human skin. Tattoo is one of the oldest communication methods discovered by human race. When tattoo styles are considered, it is known that tattoo was also used in a way to mark people after its discovery. It has been placed on human body for decorative purposes since ancient times. It is believed that the first tattoo was used in a tribe living in Peru (South America).

The tradition of tattooing is quite ancient. It was understood from mummies that ancient Egyptians used tattoos in the 2000s BC. Apart from the Egyptian society, Britons, Gauls, and Thracians also applied tattoos. The ancient Greeks and Romans used tattoos, which they considered “an occupation pertaining to barbarians”, on criminals' and slaves' bodies. Christianity prohibited tattooing. However, the first Christians tattooed their bodies with the name of Jesus or cross designs. After a few centuries, Europeans forgot about tattoos. They encountered tattoos again in native Americans and Polynesians during the overseas trips in the late 18th century. The word “tattoo” whose variations are used in European languages derived from the Tahitian word “tautau”. Tattoos gained a wider currency especially among sailors after the early 20th century. Tattooing was commonly used to indicate romantic feelings, patriotism or piety, and it is still exercised for such purposes today. On the other hand, there are ongoing efforts to impose legal restrictions on unlicensed tattooing on account of the fact that hygiene is neglected during tattooing applications and it causes transmission of some diseases such as AIDS.

Besides being a serious occupation that requires master-level motor skills, tattooing is an impressive field of art that stimulates our most basic instincts.

Temporary tattoo is usually known as Mehndi (i.e., Indian way of decorating the body by use of powdered henna). Henna (Lawsonia inermis) is a plant that may be defined as a tall shrub or small tree of approximately 2,60 meters from the Lythraceae family and a powder prepared by grinding of dried leaves of this plant. This powder is believed to possess a healing effect in North Africa and West Asia. Therefore, it is a part of rituals of some tribes in the mentioned regions.

Henna, which was previously used to apply various designs on the body, is thought to have a calming and pain-killing effect. Additionally, it was believed that henna protected people, who applied henna on their bodies, from evil and brought them luck and fertility. However, henna has been rather used as temporary tattoo in recent years. Getting an actual, i.e., permanent, tattoo on any part of human body is a difficult and painful process. Moreover, it is also possible to experience health problems due to unhygienic conditions under which the tattoo is applied. This situation creates fear and anxiety. In addition to the mentioned facts, the challenge of removing a tattoo, if one wishes to do so, led to an increase in the popularity of temporary tattooing. Temporary tattoo is quite easy to apply to the body, unlike the permanent tattoo deposited under the skin with a needle. There are many different application methods. The easiest among them is the use of a temporary tattoo spray on the human body. It takes a maximum of 2-3 minutes to apply and it is very easy to wipe off.

Sprays utilized in the prior art contain harmful materials for human health, as well as carcinogens. Additionally, sprays in the prior art can only be used on human body, other products cannot be decorated by use of the existing sprays. Another issue is that the temporary tattoos in the prior art are visible only during daytime and cannot be seen at night.

The application numbered TR2015/01137 and titled “Temporary spray for decorating human body”, which is mentioned in a state of the art, summarizes the related invention as follows: “The invention relates to body ornament structures, their derivatives and applications and describes a product group that differentiates from similar products both in terms of the sector, application and results by way of the innovation and individual, social and economic benefits it provides. The invention differs from permanent tattoo applications, as well as all types of temporary tattoo and henna applications that are known as Mehndi and accepted as derivative product, and the product named Temporary Tattoo Home Kit with Patent application number TR 201302272, in that, besides its properties mentioned in detail in the description, it possesses a spray actuator and a valve tip with time- and dose-adjusted lens tip, thereby ensuring an economical usage by preventing the person applying the tattoo from using the dye in an uncontrolled manner, providing psychological benefits (a sense of success and accomplishment) as a result of the said economical usage, eliminating health problems (especially asthma and other respiratory tract diseases) by way of preventing excessive release of gas to the relevant environment thanks to the controlled usage, improving and developing the child psychology (particularly the motivation of self-confidence and the sense of achievement) and the artistic side of children thanks to being easy-to-use, and providing a longer lasting product; and most importantly, it has the advantages of not only being a source of social pride and motivation but also creating an added value service for domestic economy since it is an added value product with a high potential of worldwide marketability thanks to its spray actuator and valve tip with time- and dose-adjusted lens tip, unlike all derivative products across the world, and it is characterized as a product with high benefit/gain parameter.” However, the invention explained herein does not describe a method.

The need to eliminate the disadvantages in the prior art has necessitated the development of a temporary tattoo method.

### DESCRIPTION OF THE INVENTION

Developed to eliminate the above-mentioned disadvantages and bring new advantages to the related technical field, the present invention relates to the temporary tattoo method applied on human body, furniture, and vehicles by use of neon paint that is visible at night and is not harmful for human health by way of spraying.

The aim of the invention is to provide a temporary tattoo that can be easily applied, can be wiped off with use of cologne whenever desired, does not cause an allergic reaction, is quick-drying, gives the feeling of a real tattoo, and is not harmful for human health.

Another aim of the invention is to ensure that the tattoo is visible only at night by utilizing neon paint.

A further aim of the invention is to guarantee that the tattoo can be easily wiped off the skin with cologne or alcohol-based solvents and is not removed when washed with water.

Another aim of the invention is to present a method that enables application of tattoos in a few minutes.

Another aim of the invention is to ensure that it does not cause allergic reactions since it is composed of vegetable dyes.

A further aim of the invention is to provide a product that is portable and can be applied on-the-go wherever desired.

A further aim of the invention is to provide a method that allows spraying the surface to be tattooed for a short time, rather than a continuous spraying, by employing a press-release valve and ensures a cautious and meticulous application since the continuous spraying is eliminated.

Another aim of the invention is to ensure that the tattooing method can also be applied on surfaces such as furniture and vehicle.

### DETAILED EXPLANATION OF THE INVENTION

This detailed description of the temporary tattoo method subject to the invention is given only to ensure a better understanding of the subject and in a way that will not create any limiting impact.

The method applied in the invention consists of the following process steps:
Tattoo stencil is placed on the body,
Spray is shaken thoroughly and is applied to the part of the skin to be tattooed from a distance of 10-15 cm by using press-release valve,
The stencil is removed after waiting for a minute,
The excess parts that splashed around are wiped off with cologne and the process is completed.

The tattoo is wiped off with use of cologne whenever desired. The temporary tattoo method allows the tattoo to stay on the body for approximately 7-10 days. It is not removed when washed with water. It is naturally erased after 7-10 days. Since the spray that is utilized within the scope of the tattooing method contains vegetable dye, it does not pose any health problems and have any side effects. A controllable spraying and a more meticulous application are ensured by installation of a press-release valve on the spray can.

What is claimed is:

1. A temporary tattoo method applied by way of spraying neon paint on a human body, furniture, and vehicles, wherein the neon paint is visible at night and is not harmful for human health, the method comprising:
placing a tattoo stencil on a body;
thoroughly shaking the neon paint;
applying, using a press-release valve, the neon paint to the body from a distance of 10-15 cm;
removing the stencil a minute after application of the neon paint; and
wiping excess neon paint from the body with cologne.
